(19) United States
(12) Patent Application Publication
(10) Pub. No.: US 2023/0094822 A1
(43) Pub. Date: Mar. 30, 2023

(54) METHODS AND SYSTEMS FOR FACILITATING REWARDING INDIVIDUALS BASED ON FITNESS CHALLENGES

(71) Applicant: PaidWorkout Corp, Miami Beach, FL (US)

(72) Inventors: Michael Bluestein, Miami Beach, FL (US); Nicole Pekerman, Miami Beach, FL (US)

(21) Appl. No.: 17/993,233

(22) Filed: Nov. 23, 2022

Related U.S. Application Data
(60) Provisional application No. 63/247,541, filed on Sep. 23, 2021.

(51) Int. Cl. A63B 24/00; A63B 71/06

(52) U.S. Cl. CPC A63B 24/0084; A63B 71/0622; G06Q 20/3223

(57) ABSTRACT

The present invention provides a method and a system for for facilitating rewarding individuals based on fitness challenges. The method and system include steps of receiving user data, analyzing the user data to generate a user account; transmitting a plurality of active fitness challenges to the at least one user device, receiving a selection; receiving a payment corresponding to the selection; receiving user activity data and personal body data; analyzing the user activity data and personal body data to generate a normalized active score; processing the normalized active score based on at least one machine learning algorithm; validating the normalized active score; generating valid normalized active score associated with the at least one user; generating a score card; generating a reward; transmitting the reward to at least winner device; and storing the reward, the user data, the valid normalized active score, the selection, the personal body data, the user activity data, and the payment.

### FIELD OF THE INVENTION

The present invention relates generally to data processing. More specifically, the present invention is methods and systems for facilitating rewarding individuals based on fitness challenges.

### BACKGROUND OF THE INVENTION

The field of data processing is technologically important to several industries, business organizations, and/or individuals.

In the recent past, new diseases have been cropping up quite regularly. As a result, maintaining health and fitness are gaining importance. However, building momentum and providing motivation to individuals is still a problem for many. Existing techniques for facilitating rewarding individuals based on fitness challenges are deficient with regard to several aspects. For instance, current technologies do not motivate an individual to take up a fitness challenge and thrive for a fitter lifestyle based on cash rewards. Furthermore, current technologies do not eliminate individuals who cheat or use unfair means in the fitness challenge using artificial intelligence. Moreover, current technologies do not ensure fair competition by normalizing a fitness score based on the Body Mass Index (BMI) of the individual.

Therefore, there is a need for improved methods and systems for facilitating rewarding individuals based on fitness challenges that may overcome one or more of the above-mentioned problems and/or limitations.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter’s scope.

According to some embodiments, a method for facilitating rewarding individuals based on fitness challenges is disclosed. Accordingly, the method may include receiving, using a communication device, user data from at least one user device associated with at least one user. Further, the method may include analyzing, using a processing device, the user data to generate a user account. Further, the method may include transmitting, using the communication device, plurality of active fitness challenges to the at least one user device. Further, the method may include receiving, using the communication device, a selection corresponding to at least one of an active fitness challenge of the plurality of active fitness challenges order from the at least one user device. Further, the method may include receiving, using the communication device, a payment corresponding to the selection from the at least one user device. Further, the method may include receiving, using the communication device, user activity data and personal body data from at least one input device. Further, the method may include analyzing, using the processing device, the user activity data and personal body data to generate a normalized active score. Further, the method may include processing, using the processing device, the normalized active score of the at least one user based on at least one machine learning algorithm. Further, the method may include validating, using the processing device, the normalized active score based on the processing. Further, the method may include generating, using the processing device, valid normalized active score associated with the at least one user based on the validating. Further, the method may include generating, using the processing device, a score card based on the generating of the valid normalized active score. Further, the method may include generating, using the processing device, a reward based on the score card for at least one winner. Further, the method may include transmitting, using the communication device, the reward to at least winner device associated with the at least one winner. Further, the method may include storing, using a storage device, the reward, the user data, the valid normalized active score, the selection, the personal body data, the user activity data, and the payment.

According to some aspects, a system for facilitating rewarding individuals based on fitness challenges is disclosed. Accordingly, the system may include a communication device configured for receiving a user data from at least one user device associated with at least one user. Further, the communication device may be configured for transmitting plurality of active fitness challenges to the at least one user device. Further, the communication device may be configured for receiving a selection corresponding to at least one of an active fitness challenge of the plurality of active fitness challenges order from the at least one user device. Further, the communication device may be configured for receiving a payment corresponding to the selection from the at least one user device. Further, the communication device may be configured for receiving activity data and personal body data from at least one input device. Further, the communication device may be configured for transmitting a reward to at least winner device associated with the at least one winner. Further, the system may include a processing device configured for analyzing the user data to generate a user account. Further, the processing device may be configured for analyzing the user activity data and personal body data to generate a normalized active score. Further, the processing device may be configured for processing the normalized active score of the at least one user based on at least one machine learning algorithm. Further, the processing device may be configured for validating the normalized active score based on the processing. Further, the processing device may be configured for generating valid normalized active score associated with the at least one user based on the validating. Further, the processing device may be configured for generating a score card based on the generating of the valid normalized active score. Further, the processing device may be configured for generating the reward based on the score card for at least one winner. Further, the system may include a storage device configured for storing the reward, the user data, the valid normalized active score, the selection, the personal body data, the user activity data, and the payment.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform consistent with various embodiments of the present disclosure.

FIG. 2 is a block diagram of a system for facilitating rewarding individuals based on fitness challenges, in accordance with some embodiments.

FIG. 3 is a flow chart of a method for facilitating rewarding individuals based on fitness challenges, in accordance with some embodiments.

FIG. 4 is a continuation flow chart of FIG. 3.

FIG. 5 is a flow diagram of a method for facilitating rewarding individuals based on fitness challenges, in accordance with some embodiments.

FIG. 6 is a continuation flow chart of FIG. 5.

FIG. 7 is a block diagram of a computing device for implementing the methods disclosed herein, in accordance with some embodiments.

### DETAIL, DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of methods and systems for facilitating rewarding individuals based on fitness challenges, embodiments of the present disclosure are not limited to use only in this context.

### Overview

The present disclosure describes methods and systems for facilitating rewarding individuals based on fitness challenges. Further, Paid Workout, an exemplary embodiment of a software platform associated with the disclosed system herein, is a fitness motivation app where users join challenges, stay active, and top the leader boards for a chance to earn cash prizes. Further, the users may transform the way they enjoy fitness with Paid Workout. Further, the disclosed system may provide the challenges to help the users stay on track and offer friendly competition among groups with similar fitness levels and one of the best motivators of all - cash reward. Further, from spinning and running to yoga and basketball, the disclosed system may be associated with a “you do you” philosophy - any activity counts. Further, the disclosed system may take a fun, quick quiz to recommend the perfect group for the user. Further, the user may invest in himself ($2-$10) and join a challenge. Further, a wearable is the best way to earn points. Further, the top 3 places on the leaderboard earn a cash reward. Further, the disclosed system may receive health data from an individual (from multiple sources). Further, the disclosed system may apply an algorithm that equalizes it based on a user’s Body Mass Index so that each person can compete fairly no matter their size. Further, the disclosed system may run a series of stringent processes to weed out any cheaters (3 steps). Further, the disclosed system may derive an active score which is used to see who places in what rank on a leaderboard.

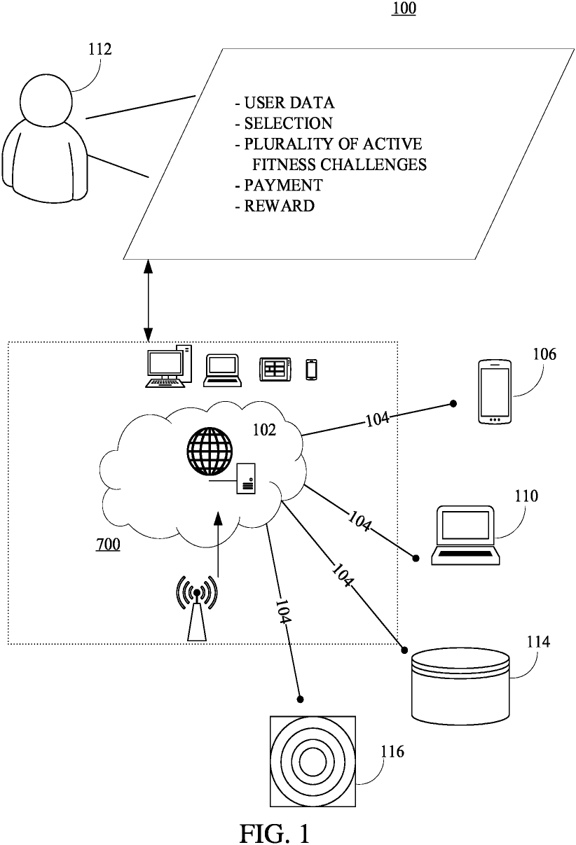

FIG. 1 is an illustration of an online platform 100 consistent with various embodiments of the present disclosure. By way of non-limiting example, the online platform 100 for facilitating rewarding individuals based on fitness challenges may be hosted on a centralized server 102, such as, for example, a cloud computing service. The centralized server 102 may communicate with other network entities, such as, for example, a mobile device 106 (such as a smartphone, a laptop, a tablet computer, etc.), other electronic devices 110 (such as desktop computers, server computers, etc.), databases 114, and sensors 116 over a communication network 104, such as, but not limited to, the Internet. Further, users of the online platform 100 may include relevant parties such as, but not limited to, end-users, fitness professionals, athletes, and administrators. Accordingly, in some instances, electronic devices operated by the one or more relevant parties may be in communication with the online platform 100.

A user 112, such as the one or more relevant parties, may access the online platform 100 through a web-based software application or browser. The web-based software application may be embodied as, for example, but not be limited to, a website, a web application, a desktop application, and a mobile application compatible with a computing device 700.

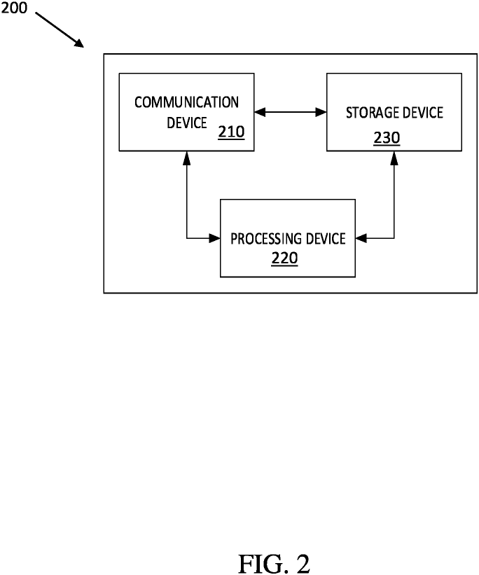

FIG. 2 is a block diagram of a system 200 for facilitating rewarding individuals based on fitness challenges, in accordance with some embodiments. Accordingly, the system 200 may include a communication device 210 configured for receiving a user data from at least one user device associated with at least one user. Further, the user data may include name, height, weight, address, email, contact number, etc. Further, the at least one user may include an individual that may want to take up a fitness challenge. Further, the at least one user device may include a smartphone, a tablet, a laptop, etc. Further, the communication device 210 may be configured for transmitting a plurality of active fitness challenges to the at least one user device. Further, the plurality of active fitness challenges may include ongoing fitness challenges. Further, the plurality of active fitness challenges may include spinning, running, yoga, basketball, etc. Further, the communication device 210 may be configured for receiving a selection corresponding to at least one of an active fitness challenge of the plurality of active fitness challenges order from the at least one user device. Further, the selection may indicate that the at least one user may want to participate in the active fitness challenge. Further, the communication device 210 may be configured for receiving a payment corresponding to the selection from the at least one user device. Further, in an instance, the payment may include a participation fee that may be $2-$3. Further, the communication device 210 may be configured for receiving activity data and personal body data from at least one input device. Further, the at least one input device may include a smartwatch, fitness band, the at least one user device, etc. Further, the user activity data may include heartrate, steps, active calories, body temperature, workout type, GPS coordinates, etc. Further, the personal body data may include age, height, weight, gender, etc. Further, the communication device may be configured for transmitting a reward to at least winner device associated with the at least one winner. Further, the at least one winner device may include the at least one user device.

Further, the system may include a processing device 220 configured for analyzing the user data to generate a user account. Further, the user account may be associated with a username and a password. Further, the processing device 220 may be configured for analyzing the user activity data and personal body data to generate a normalized active score. Further, the normalized active score may be based on BMI (Body mass index) and the user activity data. Further, the BMI may be based on the personal body data. Further, the processing device 220 may be configured for processing the normalized active score of the at least one user based on at least one machine learning algorithm. Further, the at least one machine learning algorithm may be configured for discarding the normalized active score of the at least one user that may have cheated in the active fitness challenge. Further, the processing device 220 may be configured for validating the normalized active score based on the processing. Further, the validating may include accepting the normalized active score of the at least one user that fairly competed in the active fitness challenge. Further, the processing device 220 may be configured for generating valid normalized active score associated with the at least one user based on the validating. Further, the processing device 220 may be configured for generating a score card based on the generating of the valid normalized active score. Further, the processing device 220 may be configured for generating the reward based on the score card for at least one winner. Further, the at least one winner may include the at least one user holding top three position in the scorecard.

Further, the system 200 may include a storage device configured for storing the reward, the user data, the valid normalized active score, the selection, the personal body data, the user activity data, and the payment.

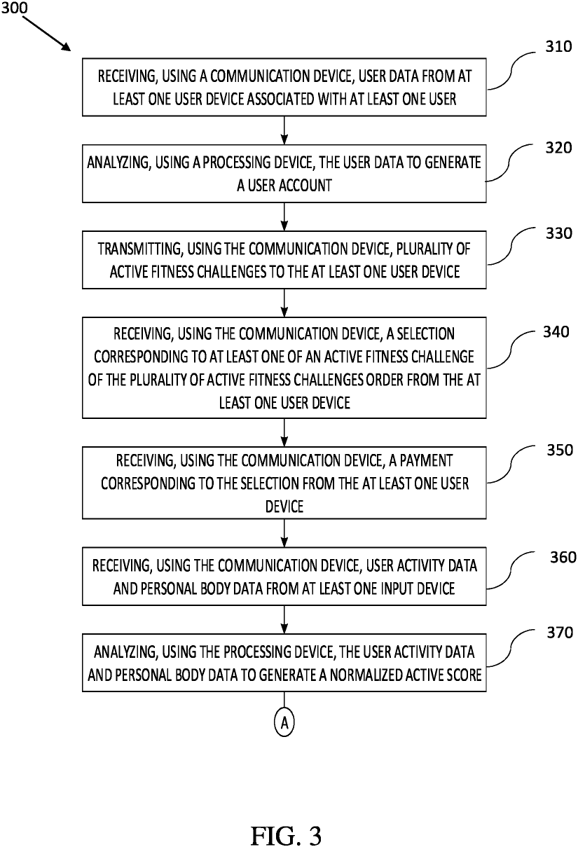

FIG. 3 is a flow chart of a method for facilitating rewarding individuals based on fitness challenges, in accordance with some embodiments. Accordingly, the method 300 may include, at 310, receiving, using a communication device, user data from at least one user device associated with at least one user. Further, the user data may include name, height, weight, address, email, contact number, etc. Further, the at least one user may include an individual that may want to take up a fitness challenge. Further, the at least one user device may include a smartphone, a tablet, a laptop, etc.

Further, the method 300 may include analyzing, using a processing device, the user data to generate a user account, at 320. Further, the user account may be associated with a username and a password.

Further, the method 300 may include transmitting, using the communication device, plurality of active fitness challenges to the at least one user device, at 330. Further, the plurality of active fitness challenges may include on going fitness challenges. Further, the plurality of active fitness challenges may include spinning, running, yoga, basketball, etc.

Further, the method 300 may include receiving, using the communication device, a selection corresponding to at least one of an active fitness challenge of the plurality of active fitness challenges order from the at least one user device, at 340. Further, the selection may indicate that the at least one user may want to participate in the active fitness challenge.

Further, the method 300 may include receiving, using the communication device, a payment corresponding to the selection from the at least one user device, at 350. Further, in an instance, the payment may include a participation fee that may be $2-$3.

Further, the method 300 may include receiving, using the communication device, user activity data and personal body data from at least one input device, at 360. Further, the at least one input device may include a smartwatch, fitness band, the at least one user device, etc. Further, the user activity data may include heartrate, steps, active calories, body temperature, workout type, GPS coordinates, etc. Further, the personal body data may include age, height, weight, gender, etc.

Further, the method 300 may include analyzing, using the processing device, the user activity data and personal body data to generate a normalized active score, at 370. Further, the normalized active score may be based on BMI and the user activity data. Further, the BMI may be based on the personal body data.

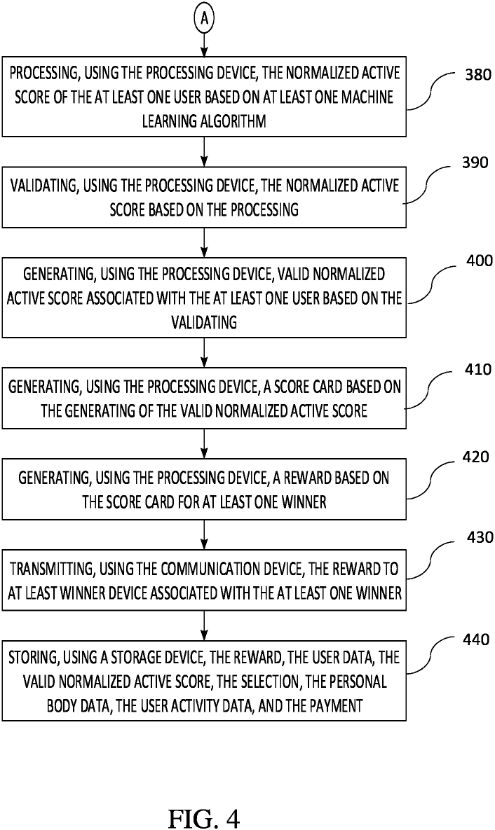

As shown in FIG. 4 (a continuation flow chart of FIG. 3), the method 300 may further include a step of processing, using the processing device, the normalized active score of the at least one user based on at least one machine learning algorithm, at 380. Further, the at least one machine learning algorithm may be configured for discarding the normalized active score of the at least one user that may have cheated in the active fitness challenge.

Further, the method 300 may include validating, using the processing device, the normalized active score based on the processing, at 390. Further, the validating may include accepting the normalized active score of the at least one user that fairly competed in the active fitness challenge.

Further, the method 300 may include generating, using the processing device, valid normalized active score associated with the at least one user based on the validating, at 400.

Further, the method 300 may include generating, using the processing device, a score card based on the generating of the valid normalized active score, at 410.

Further, the method 300 may include generating, using the processing device, a reward based on the score card for at least one winner, at 420. Further, the at least one winner may include the at least one user holding the top three position in the scorecard.

Further, the method 300 may include transmitting, using the communication device, the reward to at least winner device associated with the at least one winner, at 430. Further, the at least one winner device may include the at least one user device.

Further, the method 300 may include storing, using a storage device, the reward, the user data, the valid normalized active score, the selection, the personal body data, the user activity data, and the payment, at 440.

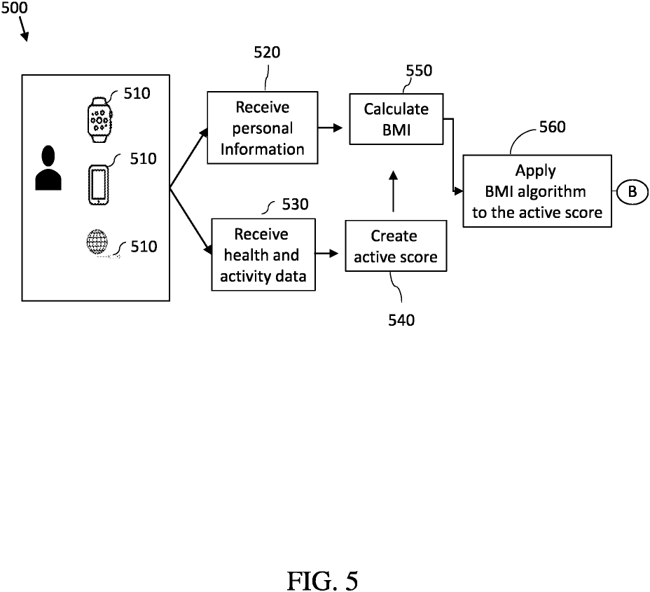

FIG. 5 is a flow diagram of a method 500 (alternative method) for facilitating rewarding individuals based on fitness challenges, in accordance with some embodiments. Accordingly, the method 500 may include receiving personal information (e.g., age, gender, weight, location, device ID) at 520 and health and activity data (e.g., heart rate, steps, minutes of exercise, active calories, workout types, GPS coordinates source of data) at 530 from a source device 510 (e.g., smart watch, smart phone, health aggregator API (application programming nterface)). Further, the method 500 may include creating an active score at 540. In some embodiments, the method may include an algorithm to create the sctive score based on active calories, steps and exercise minutes. Further, the method may include calculating body mass index (BMI) based on the personal information at 550. Further, the method may include applying a BMI algorithm to normalize the active score for fair competition at 560.

In some embodiments, the method 500 may include steps of detecting cheaters based on predetermined activities (checks). The method 500 may include a step of creating a list of potential cheaters for further review by a review process or an admin staff based on a predetermined set of checks which may include but not limited to: modification of saved data, profile data, BMI and stat data. The method 500 may include a plurality of predetermined set of checks. The admin staff or the review process may review the list of potential cheaters and make a determination on whether the potential cheaters are guilty of cheating.

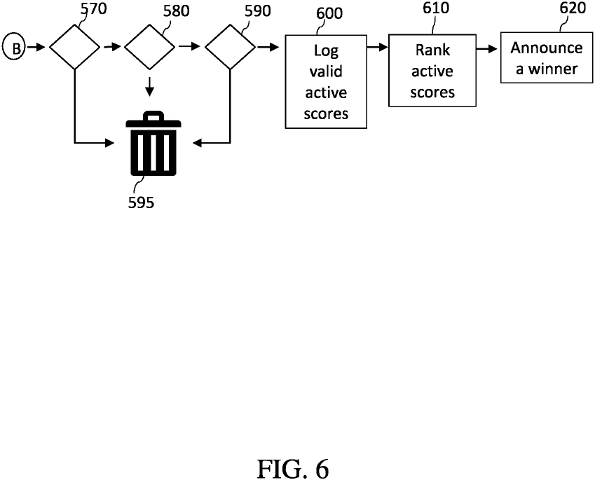

FIG. 6 is a continuation flow chart of FIG. 5. As shown in FIG. 6, in some embodiments, the method 500 may include a first stage of cheater detection 570 based on a first predetermined set of checks. Further, the method 500 may include a second stage of cheater detection 580 based on a second predetermined set of checks. Further, the method 500 may include a third stage of cheater detection 590 based on a third predetermined set of checks. The first, second, third predetermined set of checks can be different set of checks and may include but not limited to: modification of saved data, profile data, BMI and stat data. In some embodiments, the the first predetermined set of checks may include blacklisted sources that may be predetermined and the first stage of cheater detection 570 may exclude the active scores associated with the blacklisted source. The second predetermined set of checks may include amount of calories and steps per minutes and the second stage of cheater detection 580 may use an algorithm to determine if the amount of calories and steps per minutes are outside of nomal bounds. Th third predetermined set of checks may include heart rate data and the third stage of cheater detection 590 may apply an algorithm to determine if the heart rate data is manipulated.

In some embodiments, the method 500 may further include a step of creating a list of potential cheaters that can be reviewed by a review process or an admin staff. The admin staff or the review process may review the list of potential cheaters and make a determination on whether the potential cheaters are guilty of cheating.

Further, the method 500 may include a step of discarding the active score based on the determination of guilty of cheating, the first stage, the second stage, and the third stage of cheater detection, at 595.

Further, the method 500 may include logging valid active scores for competition on a per-minute basis, at 600. Further, the method 500 may include ranking of the valid active score of all competitors for the challenge at 610. Further, the method 500 may include announcing the winner based on the valid active scores when the challenge is over at 620.

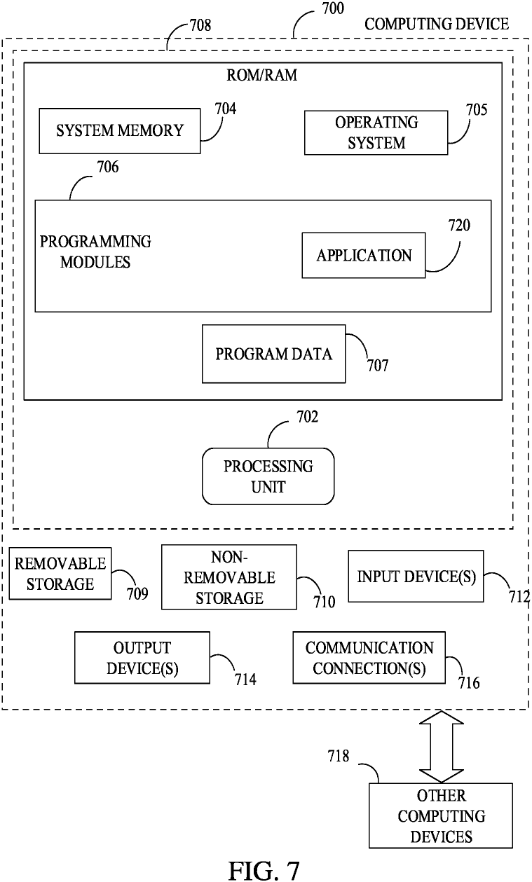

With reference to FIG. 7, a system consistent with an embodiment of the disclosure may include a computing device or cloud service, such as computing device 700. In a basic configuration, computing device 700 may include at least one processing unit 702 and a system memory 704. Depending on the configuration and type of computing device, system memory 704 may comprise, but is not limited to, volatile (e.g. random-access memory (RAM)), non-volatile (e.g. read-only memory (ROM)), flash memory, or any combination. System memory 704 may include operating system 705, one or more programming modules 706, and may include a program data 707. Operating system 705, for example, may be suitable for controlling computing device 700’s operation. In one embodiment, programming modules 706 may include an image-processing module, machine learning module, and/or image classifying module. Furthermore, embodiments of the disclosure may be practiced in conjunction with a graphics library, other operating systems, or any other application program and is not limited to any particular application or system. This basic configuration is illustrated in FIG. 7 by those components within a dashed line 708.

Computing device 700 may have additional features or functionality. For example, computing device 700 may also include additional data storage devices (removable and/or non-removable) such as, for example, magnetic disks, optical disks, or tape. Such additional storage is illustrated in FIG. 7 by a removable storage 709 and a non-removable storage 710. Computer storage media may include volatile and nonvolatile, removable and non-removable media implemented in any method or technology for storage of information, such as computer-readable instructions, data structures, program modules, or other data. System memory 704, removable storage 709, and non-removable storage 710 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 700. Any such computer storage media may be part of device 700. Computing device 700 may also have input device(s) 712 such as a keyboard, a mouse, a pen, a sound input device, a touch input device, a location sensor, a camera, a biometric sensor, etc. Output device(s) 714 such as a display, speakers, a printer, etc. may also be included. The aforementioned devices are examples and others may be used.

Computing device 700 may also contain a communication connection 716 that may allow device 700 to communicate with other computing devices 718, such as over a network in a distributed computing environment, for example, an intranet or the Internet. Communication connection 716 is one example of communication media. Communication media may typically be embodied by computer-readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer-readable media as used herein may include both storage media and communication media.

As stated above, a number of program modules and data files may be stored in system memory 704, including operating system 705. While executing on processing unit 702, programming modules 706 (e.g., application 720 such as a media player) may perform processes including, for example, one or more stages of methods, algorithms, systems, applications, servers, databases as described above. The aforementioned process is an example, and processing unit 702 may perform other processes. Other programming modules that may be used in accordance with embodiments of the present disclosure may include sound encoding/decoding applications, machine learning application, acoustic classifiers, etc.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general-purpose graphics processor-based systems, multiprocessor systems, microprocessor-based or programmable consumer electronics, application-specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer-readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid-state storage (e.g., USB drive), or a CD-ROM, a carrier wave from the Internet, or other forms of RAM or ROM. Further, the disclosed methods’ stages may be modified in any manner, including by reordering stages and/or inserting or deleting stages, without departing from the disclosure.

Although the invention has been explained in relation to its preferred embodiment, it is to be understood that many other possible modifications and variations can be made without departing from the spirit and scope of the invention.

What is claimed is:

1. A method comprising:
receiving, using a communication device, user data from at least one user device associated with at least one user;
analyzing, using a processing device, the user data to generate a user account;
transmitting, using the communication device, a plurality of active fitness challenges to the at least one user device;
receiving, using the communication device, a selection corresponding to at least one of an active fitness challenge of the plurality of active fitness challenges order from the at least one user device;
receiving, using the communication device, a payment corresponding to the selection from the at least one user device;
receiving, using the communication device, user activity data and personal body data from at least one input device;
analyzing, using the processing device, the user activity data and personal body data to generate a normalized active score;
processing, using the processing device, the normalized active score of the at least one user based on at least one machine learning algorithm;
validating, using the processing device, the normalized active score based on the processing;
generating, using the processing device, valid normalized active score associated with the at least one user based on the validating;
generating, using the processing device, a score card based on the generating of the valid normalized active score;
generating, using the processing device, a reward based on the score card for at least one winner;
transmitting, using the communication device, the reward to at least winner device associated with the at least one winner; and
storing, using a storage device, the reward, the user data, the valid normalized active score, the selection, the personal body data, the user activity data, and the payment.

2. The method as claimed in claim 1, wherein the user data includes at least one name, height, weight, address, email, and contact number.

3. The method as claimed in claim 1, wherein the at least one user includes an individual that may want to take up the active fitness challenge.

4. The method as claimed in claim 1, wherein the user account is associated with a username and a password.

5. The method as claimed in claim 1, wherein the selection indicates that the at least one user wants to participate in the active fitness challenge.

6. The method as claimed in claim 1, wherein the payment includes a participation fee.

7. The method as claimed in claim 1, wherein the at least one input device includes a smartwatch and a fitness band.

8. The method as claimed in claim 1, wherein the user activity data includes heartrates, steps, active calories, body temperature, workout type, and GPS coordinates.

9. The method as claimed in claim 1, wherein the personal body data includes age, height, weight, and gender.

10. The method as claimed in claim 1, wherein the normalized active score is based on BMI and the user activity data.

11. The method as claimed in claim 1, wherein the at least one machine learning algorithm is configured for discarding the normalized active score of the at least one user that has cheated in the active fitness challenge.

12. The method as claimed in claim 1, wherein the validating includes accepting the normalized active score of the at least one user that fairly competed in the active fitness challenge.

13. The method as claimed in claim 1, wherein the at least one winner includes the at least one user holding the top three position in the scorecard.

14. A method comprising:
receiving personal information and health and activity data from a source device;
creating an active score;
calculating a BMI;
applying a BMI algorithm to normalize the active score for fair competition;
performing a first stage of cheater detection based on a first predetermined set of checks;
performing a second stage of cheater detection based on a second predetermined set of checks;
performing a third stage of cheater detection based on a third predetermined set of checks;
creating a list of potential cheaters;
reviewing the list of potential cheaters;
determining on whether the potential cheaters are guilty of cheating;
discarding the active score based on the determination of guilty of cheating, the first stage, the second stage, and the third stage of cheater detection;
logging valid active scores;
ranking of the valid active; and
announcing a winner.

15. A non-transitory computer readable medium containing computer-readable instructions stored therein for causing a computer processor to perform operations to:
receive, using a communication device, user data from at least one user device associated with at least one user;
analyze, using a processing device, the user data to generate a user account;
transmit, using the communication device, a plurality of active fitness challenges to the at least one user device;
receive, using the communication device, a selection corresponding to at least one of an active fitness challenge of the plurality of active fitness challenges order from the at least one user device;
receive, using the communication device, a payment corresponding to the selection from the at least one user device;
receive, using the communication device, user activity data and personal body data from at least one input device;
analyze, using the processing device, the user activity data and personal body data to generate a normalized active score;
process, using the processing device, the normalized active score of the at least one user based on at least one machine learning algorithm;
validate, using the processing device, the normalized active score based on the processing;
generate, using the processing device, valid normalized active score associated with the at least one user based on the validating;
generate, using the processing device, a score card based on the generating of the valid normalized active score;
generate, using the processing device, a reward based on the score card for at least one winner;
transmit, using the communication device, the reward to at least winner device associated with the at least one winner; and
store, using a storage device, the reward, the user data, the valid normalized active score, the selection, the personal body data, the user activity data, and the payment.

16. The non-transitory computer readable medium as claimed in claim 15, wherein the normalized active score is based on BMI and the user activity data.

17. The non-transitory computer readable medium as claimed in claim 15, wherein the at least one machine learning algorithm is configured for discarding the normalized active score of the at least one user that has cheated in the active fitness challenge.

18. The non-transitory computer readable medium as claimed in claim 15, wherein the validating includes accepting the normalized active score of the at least one user that fairly competed in the active fitness challenge.

19. The non-transitory computer readable medium as claimed in claim 15, wherein the at least one winner includes the at least one user holding the top three position in the scorecard.

20. The non-transitory computer readable medium as claimed in claim 15, wherein the user account is associated with a username and a password.
